(19) United States
(12) Patent Application Publication
(10) Pub. No.: US 2005/0220742 A1
(43) Pub. Date: Oct. 6, 2005

(54) Compositions and methods for maintaining eyelid hygiene

(76) Inventor: Ed Van Breen, Manchester Center, VT (US)

Correspondence Address: PALMER & DODGE, LLP;KATHLEEN M. WILLIAMS, 111 HUNTINGTON AVENUE, BOSTON, MA, 02199, US

(21) Appl. No.: 10/816,513

(22) Filed: Mar. 31, 2004

(51) Int. Cl. A61L 9/04; A61K 7/04

(52) U.S. Cl. 424/70.1

(57) ABSTRACT

The present invention is directed to novel compositions and methods effective for maintaining eyelid hygiene, e.g., therapeutic treatment and prophylaxis. The present invention provides methods and compositions that are compliance-enhancing and useful for daily prophylaxis, which involve the easy and safe application of a foam directly to the eyelid in controlled doses effective for maintenance of eyelid hygiene.

### BACKGROUND OF THE INVENTION

“Dry eye” is the world's most common eye disease. “Dry eye” indicates the lack of quantity and/or quality of the tear film. It is described as epidemic and pandemic.

The tear film is a dynamic unit composed of an inner, middle and outer layer. The combined three layers are approximately 3 microns in total thickness, wherein a slight change in the composition of any part of the unit will cause the entire tear film to rapidly deteriorate. A lack of tears leads to a lack of nutrients and oxygen to the cornea.

Manifestations of dry eye may initially occur as ‘discomfort’ in the eye, including pain from irritation, sandy gritty sensation, loss of night vision, and excessive watery eyes, and often results in contact lens drop out, or Lasik surgery dissatisfaction. When left untreated, the consequences of dry eye can be severe, and even result in loss of vision (e.g., from desiccation of the corneal epithelium, ulceration and perforation of the cornea, or an increased incidence of infectious disease).

There are two main causes for dry eye.

1) A Lack of Tear Production (“Tear-Deficiency Dry Eye”) Which is Mainly Congenital.

Tear-deficiency dry eye can be further classified as related to the “Sjögren syndrome” or “Non-Sjögren”. Sjögren syndrome is a chronic disease in which white blood cells attack the moisture-producing glands, including those that produce tear film. The non-Sjögren syndrome related type of dry eye syndromes are those where the eye does not produce tears because of disorders of the tear gland; for example, vitamin A deficiency, contact lens wear, and diabetes and eye infections.

2) A Premature Evaporation of the Tear Film (“Evaporative Dry Eye”).

The primary manifestation of evaporative dry eye is a dysfunction of the outer, lipid layer. Normally, evaporation of the entire tear film is prevented by the thin, oily, outer layer, which contains the ‘watery’ parts of the overall tear film. This sub-micron lipid layer floats on top of the entire tear film as 1/70^{th} of the total 3 microns, is similar in thickness to a sub micron film of a soap bubble and also functions similarly. Puncture a soap bubble and the air will escape. A dysfunction of the lipid layer causes the tear to evaporate, to “break up”. This can be measured in seconds and is called tear break up time (“TBUT”). This critical, but extremely thin and fragile, lipid layer is produced by tiny glands located outside the eye, in the skin of the eyelid.

The primary manifestations of the dysfunction of the lipid layer (as well as dry eye) are all associated with inflammation (e.g. anterior and posterior blepharitis/Meibomian gland dysfunction, hordeolum, sty, and rosacea).

The etiological factors of all these inflammations are an overgrowth of bacteria (and parasites) and their toxic waste. These bacteria not only cause the lipid tear film to dysfunction, but they also destroy and block the very lipid-producing infrastructure by attacking the glands at their roots in the eyelid skin. Unfortunately, the particular types of bacteria and parasites that cause the inflammation/infections are common. The chance of having these on the eyelids is near 100%. In themselves, they are not dangerous, but it is the overgrowth and their toxic waste on the lid margin and the eyelashes that must be avoided. Allowing the bacteria and parasites to fester, nest and proliferate must be prevented, especially if one is diagnosed as a dry eye sufferer, or if there is an increased risk of having dry eye symptoms such as Lasik patients, and contact lens wearers.

With both causes of dry eye directly related to bacterial infections, it is therefore no surprise that critical (twice daily) eyelid hygiene is prescribed by physicians and recommended by health institutions worldwide. (For the life of the patient, because, as mentioned, dry eye is chronic, with no known cure and will only worsen with age).

And herein lies the problem. People tend to wash their face, but not their eyelids. Even though twice daily eyelid hygiene is so critical, there is no compliance. Due to a lack of alternatives, typically “baby shampoo” is recommended. This current “prescription” for eye hygiene has significant non-compliance issues. For example, practical applications of baby shampoo are confused by the numerous questions that remain unanswered with respect to effective use of the shampoo as a treatment for eyelid disorders. By way of illustration, how much water is to be added, when and how to apply, and how often is safe to apply? Clearly there is no standard dosage for this ‘treatment’. Thus, the baby shampoo is mixed with cocktails of warm or hot water in many different ratios, and the solution is then applied with non-sterile, dubious applicators ranging from sticks with cotton tips to unsanitary wash cloths. Thus not only endangering the patient, but also virtually ensuring little, if any, twice-daily compliance.

Also available are expensive commercial “eyelid scrubs”. These commercial eyelid “scrubs” can come in two forms; either impregnated, pre-moistened towelettes/pads, or as bottled cleansers. Bottled cleansers are applied to a non-sterile applicator and are then applied in undefined, non-standardized dosage foams created by the agitation of the applicator infused with a cleanser composition.

With only these “alternatives”, it is not surprising that there is little or no compliance to eyelid hygiene. It is further evidenced by way of commercial sales for eye care products, which shows a clear indication that people simply do not clean their eyelids: In contrast to the US eye care market for eye drops (excluding contact lens solutions) of $1 billion, current eyelid scrubs show less than $8 million in retail sales.

In addition to compliance issues, the existing cleanser compositions often overlook the special characteristics of the periocular skin. The periocular skin is more penetrable, is thinner, and is more fragile than other parts of the skin, and is in constant motion. In fact, the eyelid scrubs are typically high pH solutions and are detrimental to the periocular skin on a regular basis, because they could create further pathways for pathogens. As such, the eyelid scrubs can be detrimental to the skin, not just because they are abrasive to the skin through their applicator, but also through their composition, if used on a daily, high frequency basis for treatment of ocular disorders.

Synopsis of background: The etiological factors of the most common eye disease, dry eye, are the overgrowth on the eyelid of common bacteria and parasites as well as their toxic waste. Not only do these organisms dysfunction the tear film, they destroy the tear-producing infrastructure. Since dry eye has no known cure, optimization of the infrastructure is critical. For that reason it is deemed vital to continuously prevent any festering and proliferation of bacteria and parasites on the eyelid. Twice daily eyelid hygiene is prescribed as critical. However, due to a lack of a simple method and composition there is very little compliance and thus, dry eye is now as pandemic and epidemic.

### SUMMARY OF THE INVENTION

At present, there exists a need for a simple and easy method of cleaning an eyelid that provides the correct and defined dosage in the form of a foam, with no significant margin of error, and reduced risk of poking of the eye, e.g., with towelettes, Q-tips, or washcloths.

The present invention is directed to novel compositions and methods effective for maintaining eyelid hygiene, e.g., therapeutic treatment and prophylaxis. The methods and compositions disclosed herein are compliance-enhancing and useful for daily prophylaxis. These methods involve the easy and safe application of controlled foam directly to the eyelid in controlled doses effective for maintenance of eyelid hygiene. Thus, disruption of the delicate top layer of periocular skin around the eye by application of an undefined foam cleanser composition to the eye is avoided, as well as the need for dangerous applicators that could poke, scratch, or infect the eye. In this way, the present methods represent significant steps for increased compliance in the daily maintenance of eyelid hygiene.

### DEFINITIONS

The invention will be described with reference to following definitions that, for convenience, are collected here.

The term “controlled concentration” is defined as a characteristic of a mixture where the ratio of active ingredient(s) to non-active ingredient(s) is controllable at a prescribed level, and therefore definitive amounts of the mixture, and ingredients contained therein, can be delivered/distributed. Such a characteristic is useful in providing controllable dosage regimens (i.e., improving predictability of the dose delivered).

The term “controlled concentration foam” is defined as a foam formulated as a controlled concentration mixture of active ingredient to non-active ingredients, e.g., deionized water. The controlled concentration foam is in contrast to a liquid solution that requires further preparation, e.g., dilution and/or agitation to create a foam prior to application to the eyelid.

The term “cleaning an eyelid” is used herein to describe the act of significantly reducing the amount of dirt, debris, or otherwise undesired material, e.g., bacteria, from an eyelid.

The term “direct application” is used herein to describe the application of a cleanser composition to a subject, e.g., an eyelid of a subject, with no additional processing or preparation of the cleanser, e.g., no manual foaming or lathering, prior to application to the eyelid.

The term “dispensing” is defined as the act of delivering a cleanser composition to an applicator that has not been stored in direct contact with an applicator, e.g., in contrast to commercially available eyelid scrubs where the sponge is stored in direct contact with the cleanser liquid.

The term “dry eye” is known in the art as a condition of a subject that has a lack of quality and/or quantity of tears. Dry eye is often an age related disease. Meibomian gland dysfunction is the most frequent cause of dry eye and manifests itself in such forms as encrustation of the eyelid margins, sty, hordeolum or other inflammation of the connective tissue. Meibomian gland dysfunction is commonly linked with ocular rosacea, blepharitis, and other inflammation of the eyelids. All of these causes of inflammations of the skin are related to bacterial infection.

The term “eyelid” as used herein, includes the ocular surface, both the interior and exterior surfaces of the eyelid, the eyelid margin, the glands in and around the eyelid margins, the hair follicles of the eye, the eyelashes, and the periocular skin surrounding the eye. A front and side expanded view of the eyelid is shown in FIGS. 1A and 1B, respectively.

The term “eyelid disorder” is defined as a disorder that results in inflammation of tear producing glands or inflammation of the lipid producing glands that are located in the eyelid. Exemplary eyelid disorders include, but are not limited to proptosis, ectropion, entropion, incomplete blinking, pterygia pingueculae, conjunctivochalasis, encrustation of the eyelid margins, sty, hordeolum or other inflammation of the connective tissue, and nocturnal lagophthalmos.

The term “localized and sustained massaging”, as used herein, defines a manner of agitation of an eyelid of a subject. The massaging is focused on the eyelid for an amount of time sufficient for cleaning an eyelid, and results in significant agitation of the glands of the eyelid. This term is distinguishable from the incidental agitation of the eyelid associated with, for example, washing the entire face including the eyelid. In certain embodiments, the massaging is sustained for at least 5 seconds and possible for 10-30 seconds.

The term “ocular disorder” as used herein, includes ocular surface disorders, periocular skin disorders, and eyelid disorders, and particularly includes dry eye and symptoms related thereto. Exemplary ocular disorders include, but are not limited to dysfunctions of the tear film, inflammation of the eyelid dermis.

The term “sponge” as used herein includes all absorbent materials such as pads, swabs, tissues, Q-tips, washcloths, or fiber applicators of any kind that may be used to induce foaming and/or used as an applicator for an eyelid cleanser.

The term “transiently stable foam” is used herein to define a foam that maintains its foam nature for a sufficient amount of time as to be useful in the application to an eyelid of a subject. A transiently stable foam need not be present in the form of a foam indefinitely, but rather only as long as needed to provide a subject sufficient time to apply the dispensed foam to the eyelid.

The term “treatment” as used herein is defined as prophylactic treatment (e.g., daily preventative use) or therapeutic treatment (e.g., a single treatment or a course of treatment) of a subject with an ocular disorder, which results in the reduction, alleviation, or elimination of at least one symptom of an ocular disorder.

Methods and Compositions

Maintaining the health of the eyelid and surrounding tissue is a critical step in improving the function of the tear and lipid producing glands of the eyelid. In fact, it is not the type of bacteria, nor the quantity, but their ability to penetrate skin that causes ocular problems. Healthy skin is less penetrable by infection or infestation. FIG. 2 shows the entrance of bacteria through a skin portal, e.g., a follicle. As such, the present invention is intended to emphasize the maintenance of eyelid hygiene through prophylaxis in addition to treatment using the compositions and methods of the present invention. The present methods, which involve localized and sustained massaging of the eyelids, assist in the removal of any overgrowth of common bacteria and parasites, including the removal of the excrement released by these organisms that frequently causes the dysfunction of the tear producing glands as well as infection and inflammation of part of the eyelid (i.e., which in turn can further accelerate the cycle of tear film dysfunction).

Accordingly, the invention is directed to a cleanser composition in the form of a controlled concentration foam and it is suitable for direct application to an eyelid of a subject effective for maintaining eyelid hygiene. The foam may be generated in a substantially sponge free environment. In addition, the cleanser composition may be specifically formulated for the treatment of an ocular disorder, e.g., an ocular disorder selected from inflammation of tear producing glands or inflammation of the lipid producing glands.

The invention is further directed to a method of cleaning an eyelid of a subject or to treat ocular disorder in a subject. The method comprises the steps of providing a dispensing apparatus containing a cleanser composition, dispensing a controlled concentration of eyelid cleanser composition from the dispensing apparatus in the form of a transiently stable foam, applying the foam to the eyelid, and agitating the eyelid by localized and sustained massage of eyelid foam onto the eyelid. The foam maybe dispensed onto a fingertip and the fingertip is used to agitate the eyelid. The subject in need of treatment may have been diagnosed previously.

Furthermore, the dispensing apparatus may deliver the transiently stable foam to an applicator, e.g., a finger, utilizing a pump mechanism or a squeeze mechanism. It would also be understood by the ordinarily skilled artisan that the methods described above may utilize the controlled concentration foam in combination with any mechanically abrasive cleaning technique, for example, commercially available eyelid scrubs.

The cleaning agent of the cleanser composition may be any aqueous solution that can be formulated to form a transiently stable foam in a controlled concentration, provided that the composition is not significantly deleterious to the comfort or health of the eye and/or detracts from the compliance of use. For example, the cleanser composition may be an aqueous formulation formulated with sufficient additives to produce transiently stable foam from a dispenser engineered to produce a controlled concentration foam.

The periocular skin is distinct in nearly every important function from the rest of the body and requires special attention, especially since it is the first to manifest age related changes. At the same time, this distinct periocular skin also provides accommodation for most all of the important tear producing glands that are responsible for the tear film. (e.g., the Meibomian glands, e.g., the glands of Zeiss, Moll, and Kraus). Therefore, the present cleanser composition preferably is formulated to account for the distinct characteristics of the periocular skin. These characteristics include, but not limited to, the lower lipid count, the increased fragility as compared with other areas of skin, the lower layer count in the corneum stratum, the higher rate of exfoliation, the warm and moist environment which is conducive to infection, the age-related changes, and the fact that it represents the area where most all the tear producing glands are located.

The cleanser composition may contain the following components:
an anti-inflammatory agent in the range of about 0.01 to 7.7% by weight, selected from the group consisting of zwitterionic organic compound derivatives, e.g., ether analogs of N-(2-hydroxyl ethyl)piperazine N′(12-propane sulfonic acid) (HEPES), urethane derivative of HEPES, or aliphatic esters synthesized from piperazine-N′ propane sulfonic acid, e.g., an ester of HEPES selected from the group consisting of acetate ester, oleate ester, and linoleate ester or a long chain aliphatic ester of HEPES;
a pH-control agent and antioxidant agent in the range about 0.05-7.5% by weight selected from the group consisting of a carboxylic acid derivative of propane;
a first tissue healing agent in the range of about 0.01 to 1% by weight selected from the group consisting of allantoin and panthenol;
a first water soluble surfactant present in the range of about 0.02-20% by weight, eyelid first surfactant selected from the group consisting of sodium laureth sulfate, potassium lauryl phosphate polysorbate 60, potassium tridecyl phosphate polysorbate 60, potassium lauryl phosphate and potassium tridecyl phosphate, e.g., sodium laureth sulfate in the range of about 0.3-20% by weight;
a second water soluble surfactant in the range of about 0.01 to 7.0% by weight, which also aids in lipid replacement selected from the group consisting of disodium lauroamphodiacetate and linoleamidopropyl PG-diammonium chloride phosphate;
a skin conditioning and antibacterial agent comprising a phospholipid essential fatty acid in the range of about 0.1.to 5.0% by weight;
a microbiological preservative present in the range of about 0.01-5.7% by weight and selected from the group consisting of phenoxyethanol and diazolidinuyl urea propylene glycol/methyl-propyl parabens; and
a viscosity regulating agent in the range of about 0.1 to 6% by weight selected from one of the alkali metal salts of hydrochloric acid.
   The composition may further comprise a solubilizer in the range of about 0.01 to 5% by weight, e.g., a polyoxyethylene derivative of a fatty acid ester of sorbitol, and/or a second preservative selected from the group consisting of diazolidinyl urea propylene glycol, and methyl-propyl paraben, and/or a third surfactant of sodium borage-amidopropyl hydroxyphosphate.

The present composition may employ HEPES derivatives which are pharmacologically active as anti-phospholipase and anti-inflammatory compounds, specifically where the active ingredients are certain long chain esters of selected zwitterionic compounds based on N-substituted taurine, e.g., aliphatic esters of HEPES. Generally, the useful HEPES derivatives of the invention may be produced by catalytically reacting an alkali metal salt of HEPES with an alkyl-substituted, saturated or unsaturated, aliphatic salt, such as methyl oleate, methyl linoleate, methyl palmitate, methyl stearate, methyl myristate, and methyl behenate. The reactants are reacted in equimolecular amounts, carried out either with or without a non-aqueous solvent, such as acetone, and at a temperature range of 0° C. to the chosen solvent's reflux temperature.

The purification of the crude ester is carried out by means of crystallization in an organic solvent, dissolved in methanol, and recrystallized. U.S. Pat. No. 6,114,337, granted Sep. 5, 2000, provides detailed synthesis examples of the zwitterionic organic compounds useful here, including ether analogs and urethane derivatives of HEPES.

Commercial Applications

The methods and compositions of the invention find numerous commercial applications that could beneficially utilize the compliance enhancing methods and compositions for eyelid hygiene. Consequently, the invention includes a kit for maintaining eyelid hygiene, e.g., treatment of an ocular disorder, in a subject. The kit includes a dispenser that is capable of generating a transiently stable foam from a cleaning agent in an aqueous solution, and instructions that set forth: (1) concentrations of eyelid cleaning agent in eyelid aqueous solution formulated to generate a transiently stable foam, (2) how to use the dispenser to generate a transiently stable foam, and (3) a method of application to maintain eyelid hygiene of a subject. The kit may further comprise a cleaning agent formulated to generate a transiently stable foam and/or an applicator, e.g., a sponge. The cleaning agent may be present in a pre-measured amount. The preferred dispenser is an airless foaming device, e.g., a mini airless foamer.

The kit may be used for maintaining eyelid hygiene in a subject, e.g., treatment of an ocular disorder in the subject selected from the group consisting of inflammation of tear producing glands and inflammation of the lipid producing glands. The kit comprises a controlled concentration of the cleanser composition that is formulated to generate a transiently stable foam, and a dispenser that is capable of generating the transiently stable foam from eyelid cleanser composition, e.g., an airless foaming device. The kit may optionally be packaged with instructions for use in maintaining eyelid hygiene. The cleanser composition may be present in the kit within the dispenser and/or the kit may further comprise an applicator, e.g., a sponge.

### BRIEF DESCRIPTION OF FIGURES OF THE INVENTION

FIGS. 1A and 1B depict front and side expanded views of the eyelid, respectively.

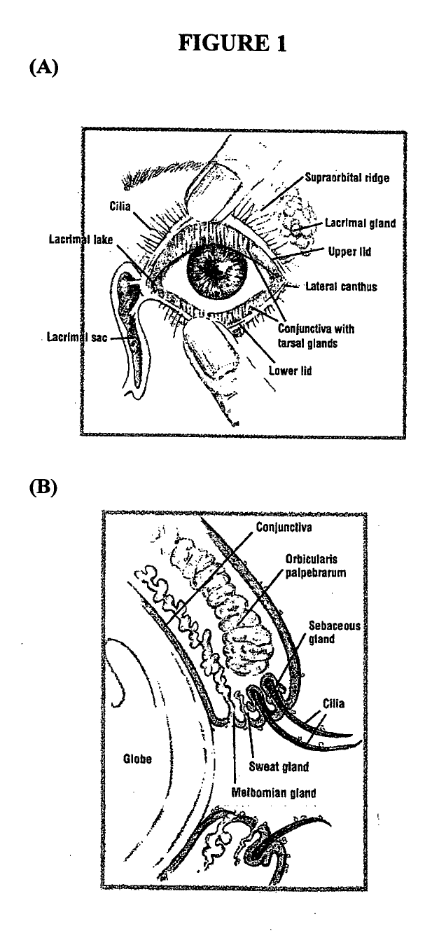

FIG. 2 depicts the entrance of bacteria through a skin portal, e.g., a follicle.

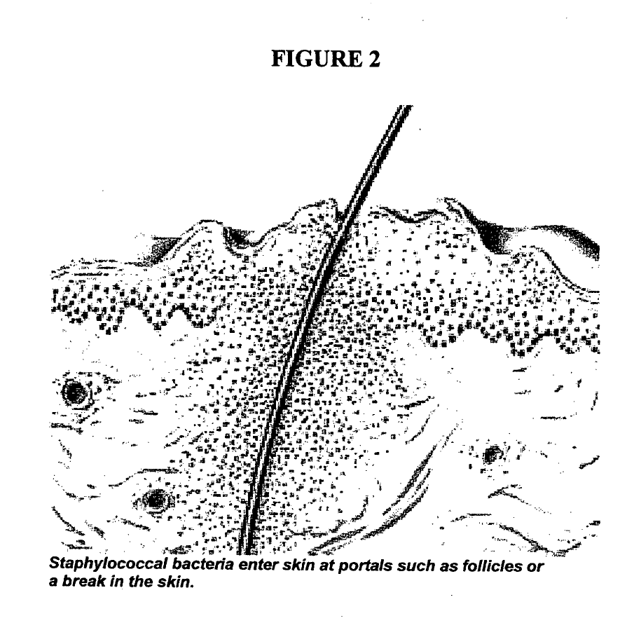

FIG. 3 depicts (a) a typical six-legged parasite present on the eyelid, (b) 3 parasitic organisms as they burrow into the eyelid, and (c) a typical eight-legged parasite present on the eyelid.

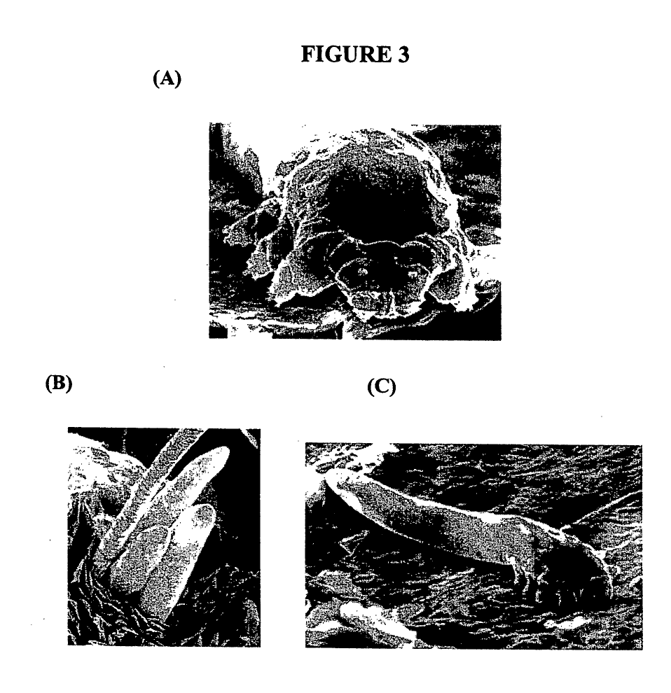

### DETAILED DESCRIPTION OF THE INVENTION

Optimization of the tear film and tear film infrastructure should include an effective method of cleaning the eyelid margin and the periocular skin. Debris causes problems not merely by blocking the tear ducts; it may also create a breeding area for pathogens close to the warm and moist environment of the eye. A method of frequently cleaning the eyelid must be easy and practical to enhance compliance.

As described above, the method of cleaning an eyelid of a subject includes the steps of providing a dispensing apparatus containing a cleanser composition and dispensing from the dispensing apparatus a controlled concentration of the cleanser composition in the form of a transiently stable foam. The dispensing apparatus can be any device that delivers a cleanser composition in the form of controlled concentration foam. However, it should be understood that, in contrast to commercially available eyelid scrubs where the sponge is stored in direct contact with the cleanser liquid, a dispenser useful in the methods of the invention is one in which the cleanser liquid, e.g., cleanser composition of the present invention, is not stored in direct contact with an applicator. For example, the dispensing apparatus may be a device that has a container portion for containing the liquid cleanser composition (or liquid cleaning agent and a separately contained aqueous portion), an induction spout that acts to draw the liquid cleanser from the container upon actuation, and a foaming portion attached to the induction spout that creates a controlled concentration foam from the liquid composition received from the induction spout. The induction spout may be actuated by a pump or a squeeze mechanism. A preferred dispenser is an airless foamer, e.g., a mini-airless foamer.

The cleanser compositions of the invention may include any aqueous solution that contains sufficient additives, e.g., surfactants or additives with surfactant-like behavior, to produce a transiently stable foam. For example, the cleanser composition may comprise any detergent that is non-deleterious to or non-harmful on the eye (particularly at the concentrations used for maintaining eyelid hygiene). For example, baby shampoo is thought in the art to be non-irritating to the eye. The compositions of the invention include detergents that may, at certain, e.g., higher, concentrations, cause damage or discomfort to eye, but at the concentrations in the cleanser composition or foam for use in maintaining eyelid hygiene, they do not substantially interfere with the normal function of the eye. Such compositions may be prepared by standard methods known in the art of formulation.

Daily cleaning of the tear producing glands is best, especially given that the quantity and quality of performance of these glands diminish daily if untreated. As such, the cleanser composition may be formulated so that application to the eyelid does not substantially damage the skin of the eyelid, even with frequent, e.g., daily, application. Furthermore, the cleanser compositions of the invention may be formulated for any desired property, e.g., substantially non-irritating, maintenance of pH of the eye, improved ability to remove dirt and debris, and/or to increase the stability of the controlled concentration foam.

The controlled concentration foam may be prepared by generating a foam from an aqueous solution that contains sufficient additives, e.g., surfactants or additives with surfactant-like behavior, to produce a foam that is transiently stable. The controlled concentration foam provides a standardized, substantially invariable, and predefined amount of cleaning agent in a given amount of foam thus, improving the dosing regimen for maintaining eyelid hygiene. Moreover, once generated, the foam is suitable for application directly to the eyelid of subject, with the advantage that the dose of the cleaning agent is well-defined, i.e., controlled, to assist in the process of accurate prescription. This is specifically in contrast to a liquid solution that requires further preparation, e.g., dilution and/or agitation to create a foam prior to application to the eyelid. Additionally, the chosen dilution ratio may be customized based on the desired application, i.e., more concentrated for applications that require increased/enhanced cleaning.

The foam should be transiently stable in order to be useful. The foam need not be present in the form of a foam indefinitely; rather, the foam needs to be stable only as long as needed to provide a subject sufficient time to apply the dispensed foam to the eyelid. The stable foam is useful in gently removing dirt and debris from the eyelid and penetrating between the eyelashes and into the hair follicle, which are known to catch debris. Additionally, a stable foam which is applied independent of a sponge applicator contributes to the improved effectiveness of the present invention by introducing the step of massaging the eyelid, which is more effective than the rubbing that is performed by the sponge applicators and is more beneficial for the tear glands.

Application of the foam to the eyelid of a subject may be by self-administration or by a trained professional, e.g., a doctor. More importantly, the application of the foam may be direct; e.g., it may be applied with a fingertip directly to the eyelid. In contrast to known methods of cleaning an eyelid which involve manual foaming or lathering, either with or without the agitation of a sponge, the present invention requires no additional processing or preparation of the cleanser prior to application to the eyelid. The advantage of eliminating this processing step ensures the presence of a standardized amount of cleaning agent in the resulting foam, i.e., the use of a controlled concentration foam.

The elimination of the need for applicators improves compliance by reducing the dangers associated with the applicator, such as the risk of poking or scratching of the eye with the applicator or introducing bacteria to the eye through the use of non-sterile applicators. Eliminating the applicator may have an additional economic advantage, i.e., eliminating the cost of an additional component of the treatment protocol. Moreover, with methods which require an applicator to generate a foam, the foam generation is less efficient, resulting in a reduction in the quality and amount of effective foam. However, one could apply a controlled concentration foam to an eyelid with a sponge or applicator. For example, the controlled concentration foam could be dispensed onto a sponge or applicator which would eliminate the foaming/lathering step required with known liquid cleanser compositions and ensure the delivery of a defined dosage to the eyelid.

Agitating the eyelid by localized and sustained massaging of the foam onto the eyelid improves the removal of dirt and debris from the eyelid as compared with known methods. Massaging is sustained for a period of time sufficient to substantially stimulate the cleaning of the ducts and glands in the eyelids, e.g., stimulating the removal of pooled sebum through sufficient agitation. For example, the massaging may be maintained for at least 5-30 seconds.

The method of cleaning an eyelid may further include a rinsing step. This step preferably comprises a simple water rinse. The foam may be rinsed from the eyelid with ample water after application and massage by bringing ample water to eyelid and eyelashes, e.g., with a hand, finger or any container suitable for this purpose.

The methods of the invention are not meant to only work in isolation. In this regard, it should be noted that it is within the contemplation of the present invention that the compositions and the methods may be used in conjunction with current methods in the art in a combination therapy/treatment. For example, while not appropriate for daily use, the commercially available eyelid scrubs may be used as part of a combination treatment with the compositions and methods of the present invention. The combination treatment may be utilized in any sequential arrangement, i.e., the commercially available eyelid scrubs may be used prior to, subsequent to, or interspersed with the treatment compositions and methods of the present invention More particularly, this invention provides a kit, as described above, containing a product useful for cleaning an eyelid, optionally packaged with additional instructions for use in maintaining eyelid hygiene in conjunction with the kits of the present invention.

Accordingly, the present invention provides improved methods of cleaning an eyelid, as well as methods of treatment of ocular disorders utilizing these methods, satisfying the need for a simple, easy, and compliance enhancing method of cleaning an eyelid that provides correct and defined dosage in the form of a foam. In fact, the advantages of the present invention include, but are not limited to improved removal of dirt and/or skin debris, improved removal of pooled sebum containing the toxic waste of bacteria and parasites (as wells as their eggs) by stimulation to the eyelids, non-irritating to the eyelid, reduced poking of the eye with cumbersome applicator sponges, increased compliance, daily application without substantial damage to the eyelid, and reduction in factors contributing to the creation of a breeding ground for pathogens. Moreover, the methods of the present invention incorporate cleansing, tissue repair, preventive maintenance, and treatment properties in a practical and consumer-friendly application.

### EXEMPLIFICATION OF THE INVENTION

The present invention may be further illustrated by the following non-limiting examples.

### EXAMPLE 1

This example shows several formulations of a cleanser composition that can be used in the present invention. Preferably, the cleanser composition is placed in premixed form in a foaming dispenser bottle such as Minifoamer, available from Airspray International Inc. This generates the transiently stable foam that can be used in the methods of the invention.

Current research underscores that the physiological processes in and on the skin take place best in a slightly acidic medium. To support the skin's functions, it is therefore important to use skin care products with a physiological pH ideally close to the skin's pH of 5.5. In addition, it is important to stabilize the skin's natural protective acid mantle to optimize its ability to withstand infection. Accordingly the pH range of the composition is best kept about 5.5-6.5, inclusive. Citric acid is a preferred pH control agent.

The composition may include deionized water (20-80% by composition) sodium laureth sulfate in a concentration of 0.3-20%, Potassium C12-13 monoalkyl phosphate polysorbate 60 (0.5-15%), potassium C12-13 monoalkyl phosphate (0.5-15%), disodium lauroamphodiacetate (0.02-12%), linoleamidopropyl PG-diammonium chloride phosphate (0.01%-5.0%), sodium chloride (0.1-6%), HEPES acetate (0.01-7.7%), citric acid(0.5-7.5%) diazolidinyl urea propylene glycol/methyl-propyl parabens (0.01-5.7%), panthenol (0.01-10%), glyoxylic-diureide/allantoin (0.01-15%), polysorbate 80 (0.01-5%) and a fragrance (0.005-3%).

In one aspect, the following active ingredients are usefully combined as having proven clinical performance as an eyelid cleanser: HEPES, oleate ester as an anti-inflammatory agent (2.00 s); a pH-control agent citric acid (1.50 s); a first tissue healing agent allantoin (0.25 s); potassium C12-13 monoalkyl phosphate (a first surfactant) (5.0 s); disodium lauroamphodiacetate (a second surfactant) (4.0 s.); diazolidimyl urea propylene (a preservative) (1.00 s.); a skin conditioning and a ntibacterial phospholipid essential fatty acid (0.1-5.0 s.); and sodium chloride (7.00 s.). Optionally, for ascetic reasons, a natural fresh and clean fragrance may be included in this formulation.

The commercially available water-soluble surfactants useful with this invention include, but are not limited to: sodium lauryl sulfate (CALFOAM ES 303); potassium C12-13 monoalkyl phosphate 60 (ARLATONE MAP 230-T60); C12-14 monoakyl phosphate (ARLATONE MAP 230K40); disodium lauroamphodiacetate (MONATERIC 949-J); and linoleamidopropyl PG-chloride phosphate (PHOSPHOLIPID EFA). Furthermore, certain ingredients, such as allantoin, panthenol and citric acid, may be incorporated into the cleansing solution.

Two examples of formulations useful in the present invention are shown below in Tables I and II as Formulations A and B, respectively.

TABLE I
Formulation A

| % | FORMULATION |
|---|---|
| 69 | Aqueous extract of chamomile |
| 14 | Potassium C-12-13 monoalkyl phosphate 80, Polysorbate 80 |
| 5 | Potassium C12-13 monoalkyl phosphate |
| 5 | Disodium Lauroamphodiacetate |
| 4 | Sodium Borageamidopropyl hydroxyphosphate |
| 1 | Sodium chloride |
| 1 | Diazolidinyl urea |
| | Methlyparaben |
| | Propylparaben |
| 0.5 | Panthenol |
| 0.25 | Glyoxylic-Diureide/Allantoin |
| 0.1 | Polysorbate 80 (a polyoxyethylene fatty acid ester based |
| | on sorbitol) |
| 99.85 | |

TABLE II
Formulation B

| TRADE NAME | CHEMICAL NAME | FUNCTION | PREF. |
|---|---|---|---|
| DEIONIZED WATER | Deionized water | Aqueous solution | 60.50 |
| | | Aqueous with soothing properties | |
| CALFOAM ES 303 | Sodium Lauryl Sulfate | Surfactant cleanser | 15.00 |
| ARLATONE MAP 230- | Potassium C12-13 | Surfactant cleanser | 5.00 |
| TWEEN 60 ™ | monoalkyl phosphate | | |
| | Polysorbate 60 | Surfactant cleanser | 7.00 |
| ARLANTONE MAP | Potassium C12-13 | Surfactant and lipid replacement | 4.00 |
| 230K40 | monoalkyl phosphate | agent | |
| MONATERIC 949-J | Disodium | Surfactant and lipid replacement | 1.00 |
| | Lauroamphodiacetate | agent | |
| PHOSPHOLIPID EFA | Linoleamidopropyl PG- | Skin conditioning and | 2.00 |
| (essential fatty acid) | Diammonium | antibacterial agent | |
| | Chloride Phosphate | | |
| SODIUM CHLORIDE | Sodium Chloride | osmolarity controlling agent | 2.00 |
| HEPES, ACETATE ESTER | N(2-hydroxy ethyl) | Antioxidant and antiinflammatory | 1.50 |
| | piperazine-N′-(2-propane | | |
| | sulfonic acid), acetate salt | | |
| CITRIC ACID | Citric acid 50% Solution | Collagen building agent, | 1.00 |
| | | antioxidant and pH control agent | |
| GERMABEN 11 | Diazolidinyl urea | Optional microbiocidal | 0.50 |
| | Methylparaben | preservative | |
| | Propyl paraben | | |
| PANTHENOL | Panthenol-alcohol analog of | Wound healer, tissue repair & | 0.25 |
| | pantothenic Acid | healing, stimulator of cellular | |
| | | proliferation, antiinflammatory | |
| ALLANTOIN | Glyoxylic-Diureide | Stimulates new and healthy tissue | 0.25 |
| | | growth, healing epithelization | |
| | | Counter irritant, moisturizer, | |
| | | softens skin | |
| TWEEN 80 ™ | Polysorbate 80 (fatty acid | Solubilizer of optional fragrance | 0.25 |
| | ester based on sorbitol) | inclusion | |
| | | | 100.00 |

### INCORPORATION BY REFERENCES

The entire contents of all patents, published patent applications and other references cited herein are hereby expressly incorporated herein in their entireties by reference.

### EQUIVALENTS

Those skilled in the art will recognize, or be able to ascertain, using no more than routine experimentation, many equivalents to specific embodiments of the invention described specifically herein. Such equivalents are intended to be encompassed in the scope of the following claims.

1. A cleanser composition effective for maintaining eyelid hygiene, said cleanser composition being in the form of a controlled concentration foam suitable for direct application to an eyelid of a subject.

2. The cleanser composition of claim 1, wherein the foam is generated in an environment that is substantially free from a sponge.

3. The cleanser composition of claim 1, wherein said cleanser composition is specifically formulated for the treatment of an ocular disorder selected from the group consisting of inflammation of tear producing glands and inflammation of the lipid producing glands.

4. The cleanser composition of claim 3, wherein the ocular disorder is selected from the group consisting of dysfunctioning of the tear film, inflammation of the eyelid dermis linked to Meibomian gland dysfunction (MGD), blepharitis, acne rosacea, seborrheic dermatitis, chalazion, hordeolum, sty and any combination thereof.

5. The cleanser composition of claim 1, wherein the ocular disorder is dry eye.

6. The cleanser composition of claim 1, wherein the cleanser comprises the following components:
(a) an anti-inflammatory agent in the range of about 0.01 to 7.7% by weight, selected from the group consisting of zwitterionic organic compound derivatives synthesized from piperazine-N′ propane sulfonic acid;
(b) a pH-control agent and antioxidant agent in the range about 0.05-7.5% by weight selected from the group consisting of a carboxylic acid derivative of propane;
(c) a first tissue healing agent in the range of about 0.01 to 1% by weight selected from the group consisting of allantoin and panthenol;
(d) a first water soluble surfactant present in the range of about 0.02-20% by weight, said first surfactant selected from the group consisting of sodium lauryl sulfate, potassium lauryl phosphate polysorbate 60, potassium tridecyl phosphate polysorbate 60, potassium lauryl phosphate and potassium tridecyl phosphate;
(e) a second water soluble surfactant in the range of about 0.01 to 7.0% by weight, selected from the group consisting of disodium lauroamphodiacetate and linoleamidopropyl PG-diammonium chloride phosphate;
(f) a skin conditioning and antibacterial agent comprising a phospholipid essential fatty acid in the range of about 0.1 .to 5.0% by weight;
(g) a microbiological preservative present in the range of about 0.01-5.7% by weight and selected from the group consisting of phenoxyethanol and diazolidinuyl urea propylene glycol/methyl-propyl parabens; and,
(h) a viscosity regulating agent in the range of about 0.1 to 6% by weight selected from one of the alkali metal salts of hydrochloric acid.

7. The cleanser composition of claim 6, wherein the active zwitterionic organic compound comprises aliphatic long chain esters synthesized from piperazine-N′ propane sulfonic acid.

8. The cleanser composition of claim 6, wherein the active zwitterionic organic compound is a long chain aliphatic ester of N-(2-hydroxyl ethyl)piperazine N′(12-propane sulfonic acid) (HEPES).

9. The cleanser composition of claim 6, wherein the active zwitterionic organic compound is an ester of HEPES selected from the group consisting of acetate ester, oleate ester, and linoleate ester.

10. The cleanser composition of claim 6, wherein the active zwitterionic organic compound is an ether analog of HEPES.

11. The cleanser composition of claim 6, wherein the active zwitterionic organic compound is a urethane derivative of HEPES.

12. The cleanser composition of claim 6, further comprising a solubilizer in the range of about 0.01 to 5% by weight,

13. The cleanser composition of claim 12, wherein said solubilizer comprises a polyoxyethylene derivative of a fatty acid ester of sorbitol.

14. The cleanser composition of claim 6, wherein the first surfactant comprises sodium lauryl sulfate in the range of about 0.3-20% by weight.

15. The cleanser composition of claim 6, further comprising a second preservative selected from the group consisting of diazolidinyl urea propylene glycol, and methyl-propyl paraben.

16. The cleanser composition of claim 6, wherein the pH range is about 5.5-6.5.

17. The cleanser composition of claim 6, further comprising a third surfactant of sodium borage-amidopropyl hydroxyphosphate.

18. The cleanser composition of claim 6, wherein the pH control agent is citric acid.

19. A method of cleaning an eyelid of a subject comprising:
providing a dispensing apparatus containing a cleanser composition;
dispensing a controlled concentration of said cleanser composition from said dispensing apparatus in the form of a transiently stable foam;
applying the foam to the eyelid; and
agitating the eyelid by localized and sustained massaging of said foam onto the eyelid.

20. The method of claim 19, wherein the foam is generated in an environment that is substantially free from a sponge.

21. The method of claims 20, wherein the foam is dispensed onto a fingertip and the fingertip is used to agitate the eyelid.

22. The method of claim 19, wherein the cleanser composition is formulated such that upon application to the eyelid said cleanser composition does not substantially damage the skin of the eyelid.

23. A method of treating ocular disorder in a subject comprising cleaning an eyelid of a subject, wherein the cleaning comprises the steps of:
providing a dispensing apparatus containing a cleanser composition;
dispensing a controlled concentration of said cleanser composition from said dispensing apparatus in the form of a transiently stable foam;
applying the foam to the eyelid; and
agitating the eyelid by localized and sustained massaging of said foam onto the eyelid.

24. The method of claim 23, wherein the treatment is performed on a subject in need of treatment.

25. The method of claim 23, wherein the ocular disorder is selected from the group consisting of dysfunctioning of the tear film, inflammation of the eyelid dermis linked to Meibomian gland dysfunction (MGD), blepharitis, acne rosacea, seborrheic dermatitis, chalazion, hordeolum, sty and any combination thereof.

26. The method of claim 25, wherein the ocular disorder is dry eye.

27. The method of claim 23, wherein said transiently stable foam is dispensed utilizing a device selected from the group consisting of a pump mechanism and a squeeze mechanism and wherein said device delivers said transiently stable foam to an applicator for application to the eyelid of the subject.

28. The method of claim 27, wherein the applicator is a fingertip.

29. The method of claim 23, wherein the controlled concentration foam is used in combination with a mechanically abrasive cleaning technique.

30. A kit for maintaining eyelid hygiene in a subject comprising:
a dispenser that is capable of generating a transiently stable foam from a cleaning agent in an aqueous solution; and
instructions that set forth:
(1) concentration of said cleaning agent in said aqueous solution formulated to generate a transiently stable foam,
(2) how to use the dispenser to generate a transiently stable foam, and
(3) a method of application to maintain eyelid hygiene of a subject.

31. The kit of claim 30, further comprising a cleaning agent formulated to generate a transiently stable foam.

32. The kit of claim 31, wherein the cleaning agent is present in a pre-measured amount.

33. The kit of claim 30, further comprising an applicator.

34. The kit of claim 33, wherein the applicator is a sponge.

35. The kit of claim 30, wherein the dispenser is an airless foaming device.

36. The kit of claim 30, wherein the maintenance of eyelid hygiene comprises treatment of an ocular disorder in the subject selected from the group consisting of inflammation of tear producing glands and inflammation of the lipid producing glands.

37. The kit of claim 36, wherein the ocular disorder is selected from the group consisting of dysfunctioning of the tear film, inflammation of the eyelid dermis linked to Meibomian gland dysfunction (MGD), blepharitis, acne rosacea, seborrheic dermatitis, chalazion, hordeolum, sty and any combination thereof.

38. The kit of claim 30, wherein the ocular disorder is dry eye.

39. A kit for maintaining eyelid hygiene in a subject comprising:
a cleanser composition in a controlled concentration and formulated to generate a transiently stable foam; and
a dispenser that is capable of generating a transiently stable foam from said cleanser composition formed of a cleaning agent in an aqueous solution.

40. The kit of claim 39, wherein the cleanser composition is present in said dispenser.

41. The kit of claim 39, wherein the dispenser is designed to deliver the foam in the form of a controlled concentration foam suitable for direct application to an eyelid of a subject

42. The kit of claim 39, further comprising an applicator.

43. The kit of claim 42, wherein the applicator is a sponge.

44. The kit of claim 39, wherein the dispenser is an airless foaming device.

45. The kit of claim 39, wherein the maintenance of eyelid hygiene comprises treatment of an ocular disorder in the subject selected from the group consisting of inflammation of tear producing glands and inflammation of the lipid producing glands.

46. The kit of claim 45, wherein the ocular disorder is selected from the group consisting of dysfunctioning of the tear film, inflammation of the eyelid dermis linked to Meibomian gland dysfunction (MGD), blepharitis, acne rosacea, seborrheic dermatitis, chalazion, hordeolum, sty and any combination thereof.

47. The kit of claim 39, wherein the ocular disorder is dry eye.

48. The kit of claim 39 packaged with instructions for use in maintaining eyelid hygiene.
